(19) United States
(12) Patent Application Publication
(10) Pub. No.: US 2014/0206737 A1
(43) Pub. Date: Jul. 24, 2014

(54) Application of Clonidine Hydrochloride as an Information Intervention Agent and an Information Intervention Product and the Method of Making the Same

(71) Applicant: Beijing Zhongwei Shennong Chronic Disease Medical Reasearch Institute Co., Ltd., (US)

(72) Inventors: Mengjun YANG, Beijing (CN); Zhe YANG, Beijing (CN)

(73) Assignee: Beijing Zhongwei Shennong Chronic Disease Medical Research Institute Co., Ltd., Beijing (CN)

(21) Appl. No.: 13/746,056

(22) Filed: Jan. 21, 2013

(51) Int. Cl. A61K 9/00; B65D 25/00; A61M 37/00; B65B 1/04

(52) U.S. Cl. CPC A61K 9/009; B65B 1/04; B65D 25/00; A61M 37/00
USPC 514/401; 548/333.1; 53/473; 206/205; 604/291

(57) ABSTRACT

This invention relates to a new application of compound Clonidine Hydrochloride in a use of preparation of information intervention agent, a new preparation method of information intervention agent, and a product. The Clonidine Hydrochloride interacts with the human information receptor, therefore realizing the intervention through the Clonidine Hydrochloride information physical method, and treatment was not a chemical treatment. This invention comprises: putting the compound Clonidine Hydrochloride information package filler (3) into a container (4), closing the lid (7), sealing by a glue, attaching a hanging piece to the container (4) to form the Clonidine Hydrochloride information intervention agent (9) for clinical use. The usage of this invention is simple, convenient, safe, reliable and easy to accept. It is especially appropriate for use by senior people, unhealthy people, children, or people with disturbance of consciousness, mental retardation, and behavioral disorders. This invention can reduce the trouble of administration of the drug by the nurses, reduce their labor intensity, and save human resources.

### FIELD OF TECHNOLOGY

This invention relates to the field of chemistry, in particular to the application of a type of compound clonidine hydrochloride as an information intervention agent and an information intervention product and the method of making the same.

### BACKGROUND

The compound clonidine hydrochloride, also known as 2-(2,6-Dichloroanilino)-2-imidazoline hydrochloride, has a chemical formula C₉H₉Cl₂N₃.HCl, and a molecular weight of about 266.56. It is a white crystalline powder, odorless, soluble in water or ethanol, very slightly soluble in chloroform, and practically insoluble in ether. A known use of clonidine hydrochloride is as a chemical drug that enters the human body by oral administration or by transdermal absorption, and is excreted from the human body by metabolic decomposition. Clonidine hydrochloride is a central α2 receptor agonist, clinically used to treat high blood pressure, migraine, menopausal hot flashes, dysmenorrhea, and rapid detoxification during the opiate withdrawal. Pharmaceutical formulations of clonidine hydrochloride include tablets, injection, eye drops, transdermal patches and gel formulation agents. The known clonidine hydrochloride medications are used as a chemical treatment; the compound clonidine hydrochloride molecules enter the human body (by direct injection into the blood, oral absorption into the bloodstream, or through the skin and mucous membrane absorption into the blood), and distribute to distinctive organs, and produce pharmacological effects through chemical reactions of molecules in the human body. The use of clonidine hydrochloride as the chemical treatment has its insurmountable disadvantages. First, its side effects on the human body include dry mouth, drowsiness, dizziness, constipation, sedation, general weakness, fatigue, headache, orthostatic symptoms of the cardiovascular system, palpitations, tachycardia or bradycardia, congestive heart failure, abnormal electrocardiogram, nervousness and agitation, depression, insomnia, behavioral changes, nausea, vomiting, stomach intestinal discomfort, abnormal liver function, regression of libido, impotence and loss of libido, etc. Second, the therapeutic window is small. The effective dose that is approved for use is very small due to the side effects on the human body, which limits the effect of the treatment results. The approved maximal oral administration dose is 0.6 mg each time and 2.4 mg each day. Any dose beyond the approved maximal oral administration does can lead to intoxication. Third, the only known use is as a chemical treatment. After entering the human body, Clonidine hydrochloride is metabolically broken down or excreted from the human body, which prevents prolonged effects.

### SUMMARY

The goal of this invention is to overcome the disadvantages of the compounds clonidine hydrochloride as a chemical treatment, and to find its new use and new methods to overcome the disadvantages, thereby creating an unprecedented new product as an information intervention agent. This invention is also to present the method of making an information intervention agent from clonidine hydrochloride and the resultant product.

In order to realize the goal, the invention adopts the following technical solutions.

A method of making a clonidine information intervention product, according to an embodiment, comprises selectively adding paraffin and supplement carrier materials to clonidine hydrochloride powder based on a specific mass fraction; sealing the resultant mixture in a porous carrier material; packing the mixture in a container (with pores; and assembling a piece of hanger hardware onto the container with pores. The clonidine information intervention product can be used by hanging it near a surface of the human body, at a distance of 1-10 mm (preferably about 2 mm) to the surface of the human body (e.g., skin), preferably at the umbilicus, with the pores facing the surface of the human body (e.g., skin). According to treatment needs, the clonidine information intervention product may be used once a day for 1-3 hours, or twice a day for 1-2 hours. The intervention is achieved through a physical interaction of the clonidine information with information receptors in the human body, instead of through clonidine hydrochloride entering the human body and reacting with clonidine receptors to achieve the therapeutic treatment. In this invention, clonidine is used as an information intervention agent, not as a chemical treatment drug. After a period of time in use as the information intervention agent, the clonidine material may be used again as a chemical treatment drug after the wax is removed. When the clonidine material is prepared as a conventional agent in chemical treatment, it has the same therapeutic effect as the clonidine hydrochloride. In addition, it can be used as a synergist for the conventional preparation.

In this invention, said newly synthesized clonidine raw material powder refers to clonidine raw materials within three months of its synthesis and containing no less than 99.0 wt % of dry C₉H₉Cl₂N₃.HCl.

In this invention, said paraffin is a mixture of solid hydrocarbons from either petroleum or shale oil, suitable for use as medical wax matrix material.

In this invention, said supplement carrier material is fine particles, with diameters of 100 nm-10 micron, of crushed natural mineral tourmaline functional crystal material selected from the group consisting of lithium-, iron-, and magnesium-rich tourmaline, sodium- and manganese-rich tourmaline, calcium- and magnesium-rich tourmaline, and Bouguer tourmaline.

In this invention, said porous carrier material is selected from the group consisting of porous nonwoven fabric, porous fabric, porous paper and porous plastics. The size and density of the pores are such that the carrier material is both air permeable and leak-proof of the powder in the clonidine information intervention product.

In this invention, said container with pores can be a round box, an oval box, a box of any other shape, a bag or any other suitable container. The container has pores on one or more of its sides, wherein the pores have diameters between 0.3-3 mm.

In this invention, said hanger hardware is a support fixture that is configured to support the clonidine information intervention product. The hanger hardware can be a rope, belt, bag, rack, hook or sticker, etc.

In this invention, said clonidine information intervention product has a formulation of the clonidine raw material (1), packed and hung at a distance of 1-10 mm, preferably 2 mm, to the human skin, to prevent the direct contact of the drug substance with the skin. There is no absorption of the drug substance, and the intervention to human body is only through its information intervention.

In this invention, according to an embodiment, the method of making the clonidine information intervention product includes: based on mass fraction, weighing clonidine hydrochloride raw material 1-90%; paraffin 1-90%; supplement carrier material 1-30%, said supplement carrier material being the ultrafine particle in a diameter range of 100 nm-10 micron of the natural mineral tourmaline functional crystal materials; putting the clonidine hydrochloride raw material, supplement carrier material and paraffin into a mixer and mixing mechanically and thoroughly at room temperature and atmosphere pressure, to form the clonidine information agent packing material, at the mixing temperature of 10-25° C., preferably 20° C., and with a mixing time of 30-60 minutes, preferably 45 minutes; putting the fully mixed packing material into a powder dispensing machine, and dispensing the clonidine information agent packing material into bags made of the porous carrier material at 1-500 grams per bag, and sealing the bag by heating or other methods, to form clonidine information agent package fillers; putting the clonidine information agent packet filler into the container with pores, one packet filler per container; attaching the hanger hardware to the container. The clonidine information intervention product can be heated using a microwave oven at medium power level for 3-5 minutes prior to use for better results.

In this invention, according an embodiment, the method of making the clonidine information intervention product further include: weighing 1-200 g of paraffin particles; placing the paraffin particles in the container and then inside an oven, and heating the container to a temperature of 70° C. until the paraffin particles melts completely; weighing 1-500 g of the clonidine raw material; weighing 1-50 grams of the supplement carrier material; putting the clonidine raw material and the supplement carrier material into the melted paraffin, mixing thoroughly to embed the clonidine raw material and the supplement carrier material into the melted paraffin; putting the mixture in the oven and heating at a temperature of 70° C. for another 10-30 minutes; wrapping the mixture with the porous carrier material and compressing the wrapped mixture in a mold press; after cooling down and solidification, placing the molded product in a container with pores for packaging.

To facilitate understanding of this invention, the terms used in this invention are further explained.

The term “information intervention agent” as used herein means a non-chemical treatment drug. A chemical treatment drug is a substance which enters the human body by conventional means, such as by direct injection into the blood, oral absorption through the skin into the blood, or transdermal and mucosal absorption into the blood, wherein the substance distributes in various human organs, produces its pharmacological effects by chemical reaction with the human body, is broken down or metabolized in the human body, and is excreted. In contrast, the information intervention agent does not contact the human body nor does it enter the human body; it only uses its molecular information to regulate the body functions, and to realize intervention. Thus, treatment using an information intervention agent is novel and is based on a physical method of drug information intervention.

The term “secondary application of drugs” or “secondary use” as used herein means that the compound clonidine can be used twice or at a second time. This invention discloses the application of the information intervention of clonidine and its enhancing effect beyond common knowledge, because clonidine is not absorbed by the human body during the use in information intervention, and only the information of clonidine is used, which allows the use of the compound at a second time as a chemical treatment after the information intervention use, and does not affect the conventional chemical treatment effect of clonidine in a chemical treatment.

The term “therapeutic window” as used herein is the ratio of the effect to the side effects. The size of the therapeutic window can be understood as follows. If the affinity of a drug to its target and its side effect sites differs greatly, and the affinity to the target sites is stronger than the affinity to the side effect sites, then the drug has a large “therapeutic window”. When the therapeutic window is large enough, the dose can be increased by 5 times, 10 times, or even 50 times without serious side effects. Ideally, a developer of new drugs, doctors or patients desire that the drugs taken have the therapeutic window greater than 100 or greater than 500. One point to note is that when determining the dose of a drug for a large group of people, the differences in drug metabolism among individuals may be as high as 10 times. For some patients, a therapeutic window of only 10 times effectively equals no effective window. In fact, the conventional chemical treatment using clonidine drug has a very small therapeutic window. However, when the clonidine hydrochloride is used as an information intervention agent, the therapeutic window increases by more than 1000 folds from the therapeutic window of using clonidine in a chemical treatment.

It is contemplated that the use of clonidine hydrochloride as an information intervention agent is applicable to treatment of high blood pressure and sedation, analgesia, menopausal syndrome, withdrawal syndrome of addiction disorders, ulcerative colitis, children with short stature, children with attention deficit hyperactivity disorder, depression and others.

This invention can replace the chemical treatments, and can be used as the synergist to expand the therapeutic window of its chemical drug, enhancing the treatment effects of the chemical treatment without increasing the side effects of the drug.

This invention discloses the new use of the compound clonidine hydrochloride as an information intervention agent, discloses a new type of clonidine product and the method of making the same, and shows significant effectiveness as presented below.

First, this invention greatly expands the original clonidine hydrochloride therapeutic window in a chemical treatment. In the use of clonidine hydrochloride as the information intervention agent, no clonidine substance enters the human body, which avoids the side effects of clonidine, expands the therapeutic window from only 10 to more than 1000. The effects of clonidine treatment are enhanced while the side effects of clonidine are avoided. Meanwhile, when the conventional formulations of clonidine hydrochloride as a chemical treatment is not satisfactory, clonidine hydrochloride used as the information intervention agent can function as synergist and enhance the effect of clonidine hydrochloride as a chemical treatment without increasing its side effects.

Second, this invention can achieve secondary application of the clonidine hydrochloride. Clonidine hydrochloride, after use for one to two months as an information intervention agent, does not have diminished effect as a chemical treatment and may be used as a chemical treatment drug after wax removal, which significantly reduces the cost of treatment and increases the application value of clonidine hydrochloride. At the same time, the repeated use of clonidine reduces waste of drug, saves medical resources, and reduces environmental pollution caused by the pharmaceutical industry.

Third, this invention does not require use of a lot of accessory materials, additives, stabilizers, excipients and other non-drug ingredients used in the existing conventional pharmaceutical preparations in order to ensure the stability of the drug ingredients into the human body, and also avoids the adverse effects of these non-drug ingredients entering the human body.

Fourth, the usage of this invention is simple, convenient, safe, reliable and easy to accept. It is especially appropriate for use by senior people, unhealthy people, children, or people with disturbance of consciousness, mental retardation and behavioral disorders.

Fifth, this invention can reduce the trouble of administration of clonidine hydrochloride, reduce their labor intensity, and save human resources.

### BRIEF DESCRIPTION OF FIGURES

FIG. 1 is a schematic diagram of the package filler.

FIG. 2 is a schematic diagram of the container.

FIG. 3 is a schematic diagram of the interior of the top of the container.

FIG. 4 is a schematic diagram of the exterior of the top of the container.

FIG. 5 is a schematic diagram of the information intervention agent.

In the figures: 1, raw material, 2, porous carrier material, 3, package filler, 4, container, 5, pores, 6, plastic cover, 7, lid, 8, hanging piece, 9, information intervention agent.

### DETAILED DESCRIPTION

The following embodiments are described to assist the understanding of this invention, but the scope of this invention is not limited to the embodiments.

An embodiment for the clinical use of clonidine hydrochloride to treat high blood pressure is described below.

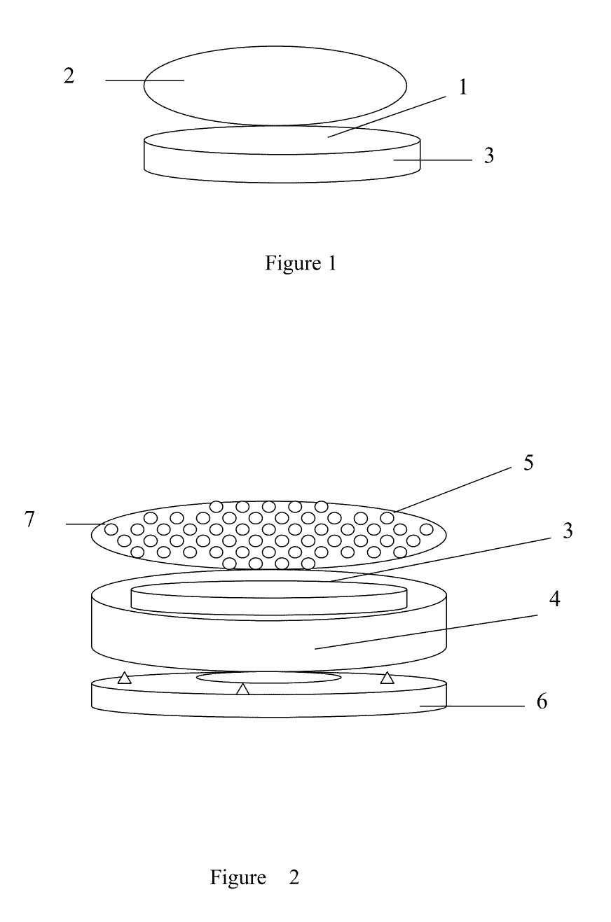

With reference to FIGS. 1 and 2, a method of making 10 clonidine information intervention products comprising weighing 20 grams of clonidine hydrochloride raw material 1 that is within three months from synthesis and meets the quality standards in the Chinese Pharmacopoeia; weighing 10 grams of paraffin (grain); weighing 1 gram of ultrafine powder of natural mineral tourmaline crystal material with particle diameter of less than 10 micron; mixing the clonidine hydrochloride raw material 1 thoroughly using a micro-mixer at a temperature of 20-25° C.; then putting the mixture in a small-dose powder dispensing machine, and dispensing the mixture into 10 bags made of the porous carrier material and sealing the bags by heating to form clonidine information package fillers 3 of 3.1 grams each; putting the clonidine information agent packet fillers 3 into 10 containers 4 with pores 5, filler 3 per container 4, wherein the container 4 is oval in shape and has a diameter of 6 cm, a height of 1 cm, a lid 7 and a number of pores 5 of 1 mm diameter on one side of the container 4; opening the lid 7, and putting the clonidine information package filler 3 into the container 4; closing the lid 7, and sealing the container 4 with an adhesive; installing the hanger hardware 8, such as a rope (belt), onto the container 4. Ten clonidine information intervention agents 9 are ready for clinical use. At the same time, another ten identical containers 4 of the same size, shape and color without the clonidine information package filler 3 are prepared and used as a placebo control.

Observation of clinical effects of the clonidine information intervention agents 9 on ten cases of clinical hypertension patients in the antihypertensive aspects are summarized as follows.

1. The Method.

This study was a single-blind, self, placebo-controlled study. The subjects observed were with mild to moderate essential hypertension (DBP: 95-114 mmHg). There were a total of 10 cases, including 8 males and 2 females, with the mean age of 50.2±9.67 years (39-62 years). The hypertension patients with the following diseases were excluded: secondary hypertension, severe respiratory disease, liver and renal insufficiency, pregnancy, depression and skin allergies. The study required that all the selected patients stop taking all anti-hypertensive drugs, and use the placebo for two weeks; at the end of the placebo phase, blood pressure measurement was made followed by 24-hour ambulatory blood pressure monitors (ABPM) (such as Spaulabs 90207 made by Spacelabs Healthcare). In a following observation phase each patient wore a clonidine information intervention agent 9 hung at his or her umbilicus, with the pore 5 of the container 4 facing the skin for 4-5 hours each time every day. On day 7, a 24-hour ambulatory blood pressure monitoring was done. If the blood pressure was not reduced to the target blood pressure range (clinic blood pressure, DBP 90 mmHg) by using once a day, the use then was changed to twice daily for three hours each time from the second week. Again, the ABPM was done at the end of the third week. If by using twice daily for three hours each time, the patient blood pressure control was still not effective, then the monitor was stopped. After the monitoring of all patients using the clonidine information agent was completed, the observation continued for two more days. On the second day, ABPM was done to observe any lasting effect of clonidine. While the ABPM was done, the clinic blood pressure (the mean value of the sitting blood pressure taken as measured three times with an interval of 2 minutes) and heart rate were measured, other responses from the user were observed, and any adverse reaction was recorded.

Results to determination: before and after comparison of the blood pressure values, according to the clinical research guidelines points of the Chinese Ministry of Health.

“Significantly effective” means: DBP≧10 mmHg and dropped to normal; DBP not yet dropped to normal, but dropped by more than 20 mmHg.

“Effective” means: DBP dropped to normal although not by more than 10 mmHg; or DBP not yet dropped to normal, but dropped by 10-19 mmHg. n the case of systolic hypertension, systolic blood pressure dropped by ≧30 mmHg.

“Ineffective”: although a decline in blood pressure is observed, the decline is below the above standards.

The total effective rate (significantly effective+effective) is statistically analyzed using paired paired t test of the blood pressures before and after using clonidine information intervention agent 9.

2. The Results.

Among the selected cases of 10 patients with primary hypertension six had their blood pressures dropped to the target range by using the clonidine information intervention agent 9 once a day. The remaining four cases had their blood pressures dropped to the target range by using the clonidine information intervention agent 9 twice a day.

After the use of the clonidine information intervention agent 9, the clinic blood pressure dropped significantly compared with before (p<0.01). The 24-hour average diastolic blood pressure (DBP), the average DBP during daytime (6 am-22 pm) and the average DBP during nighttime (22 pm-6 am) were all significantly reduced (p<0.05). The 24-hour average systolic pressure, the average daytime and nighttime systolic blood pressure were all significantly reduced (p<0.05). The heart rate did not change significantly before and after use. The day after the end of treatment, the clinic blood pressure increased slightly; the 24-hour systolic pressure increased slightly, while the diastolic pressure remained essentially unchanged. Before and after the use of the clonidine information intervention agent (9), the 24-hour ambulatory blood pressure circadian rhythm had no significant changes.

No side effects were observed.

According to an embodiment, the clinical use of the clonidine information intervention agent 9 for the treatment of heroin addiction is described below.

The clonidine information intervention agent (9) was prepared according to the following recipe:

Clonidine hydrochloride (content≧99%) 3 grams;

Paraffin wax (particle) 1 gram;

Ultrafine Tourmaline (diameter≦5 micron) 0.2 gram;

Mixing the above materials thoroughly at room temperature and atmosphere pressure; placing the mixture in a small-dose powder dispensing machine; packing the mixture in microporous air-permeable non-woven fabric bags at a net weight of 4.2 grams of mixture per bag; sealing the bags by heating; putting the bags in oval-shaped containers 4 with pores 5 on one side of the container 4, the pores 5 having a diameter of 1 mm; packing and installing hanger hardware. Before each use, the clonidine information intervention agent 9 is heated in a microwave oven at medium power for 1-2 minutes.

The comparison between the clonidine information intervention agent 9 and methadone in the treatment of heroin addiction is summarized below:

1. Materials and Methods.

1.1. Subject selection criteria: 60 participants, in the range of 18-50 years of age, male or female, and opioid dependent as diagnosed according to the DSM-IV diagnostic criteria for opioid dependence. Prior to this study, the participants had been using opioids, had significant symptoms from attempted withdrawal, tested positive for morphinein urine; and exhibited cutis anserina, sweating, dilated pupils and other positive reactions during opioids addiction tests with naloxone. The route of heroin-containing narcotics ingestion was not limited (over 60% ingested heroin-containing narcotics by insufflation and intravenous injection).

1.2. Subject exclusion criteria: candidates with serious heart disease, liver or kidney dysfunction, mental illness, serious bodily weakness, or pregnant or lactating women were excluded.

1.3. Grouping: 60 cases of participants were randomly divided into two groups according to the order of admission: the test group and the methadone control group. The test group had 30 participants, and the methadone control group had 30 participants. The difference between the general characteristics of the two groups was not statistically significant, as shown in Table 1.

TABLE 1
Comparison of general characteristics of the two groups (x ± s)

| | Clonidine | | | |
|---|---|---|---|---|
| | information | | | |
| | intervention | Methadone | | |
| | agent test | control | | |
| Description | group | group | T | p |
| Age (years) | 27.00 ± 6.01 | 27.27 ± 6.39 | 0.1676 | >0.05 |
| Weight (kg) | 53.38 ± 4.67 | 54.97 ± 5.75 | 0.15 | >0.05 |
| Time of | 2.47 ± 0.56 | 2.55 ± 0.71 | 0.50 | >0.05 |
| addiction (years) | | | | |
| Dose of | 1.04 ± 0.22 | 0.86 ± 0.13 | 2.11 | >0.05 |
| addiction (g/d) | | | | |

1.4. Test methods: the test group used the clonidine information intervention agent 9. The control group used methadone hydrochloride oral medication of 10 mg/10 ml each. Comparison method: according to the heroin-dependent self-restraining degree of 7-10 days, the treatment was designed to be 10 days long for both groups. The test group used the clonidine information intervention agent 9 once daily for six hours each time. The clonidine information intervention agent 9 was hung at the participants' umbilicus with the pores 5 facing the skin. The treatment concluded on day 10. Methadone control group was admitted 50-60 mg methadone on the first day; starting on day 2, the methadone dose was reduced by 10 mg per day each day; starting on day 6, the methadone dose was reduced by 2 mg per day each day. The treatment concluded on day 10. Supplementary drugs used in the two groups included normal doses of clonazepam tablets (clonazopam), alprazolam tablets (Alprazoli) once a day at night.

1.5. Method of Observation and Evaluation.

1.5.1. Clinical rating used indicators of withdrawal, which has 20 indicators including craving, anxiety, agitation, nausea, vomiting, hot face, chills, or alternating chills and fever, yawning, runny nose, tears, sweating, drowsiness, cutis anserina, tremors, bone and muscle pain, anorexia, abdominal pain, diarrhea, insomnia, and curled position. The rating was done daily on the day before the treatment and on each day during the treatment. The severity of withdrawal symptoms was scored as level 0-4 (five levels in total). Scoring for each participant was done at 9:00 am every day. During the process of the treatment, all participants were cooperative.

1.5.2. Recordation of adverse reactions: adverse reactions were evaluated in the withdrawal symptom measurement table, using a total of 10 indicators that include: dizziness, syncope, malaise, nausea, vomiting, sweating, dry mouth, drowsiness, blurred vision, disturbance of consciousness. The recordation was done each morning at 9:00.

1.5.3. The degree of anxiety symptoms: The Hamilton Anxiety Scale (HAMA) was used to assess in the degree of anxiety in the participants during the treatment.

1.6. Statistical analysis of the two groups: the general characteristics, withdrawal symptoms, and anxiety symptoms of the two groups, were statistically analyzed using the t test, chi-square test and Fisher's Exact Test, using suitable software such as SPSS 7.0. The t test was applied to the symptom data of the two groups, and chi-square test or Fisher's Exact Test was applied to the count data.

2. Results

2.1. Comparison in the degree of withdrawal: the difference between the test group and the methadone control group in withdrawal symptoms was not statistically significant (p>0.05). During day 1 through day 5, the test group had more severe withdrawal symptoms than the methadone control group (p<0.01). During day 6 through day 10, the test group had less severe withdrawal symptoms than the methadone control group (p<0.01). Daily changes in the withdrawal symptoms of the two groups during the 10-day treatment are shown in Table 2.

TABLE 2
Comparison of the withdrawal symptoms between
the two groups (x ± s)

| | Clonidine | | | |
|---|---|---|---|---|
| | information | | | |
| | intervention | Methadone | | |
| Days of | agent test | control | | |
| treatment | group | group | t | p |
| Before | 55.43 ± 1.91 | 56.03 ± 1.80 | 1.25 | >0.05 |
| treatment | | | | |
| Day 1 | 50.54 ± 1.33 | 5.01 ± 0.83 | 159.07 | <0.01 |
| Day 2 | 42.07 ± 1.90 | 27.78 ± 1.40 | 32.16 | <0.01 |
| Day 3 | 32.48 ± 2.00 | 19.71 ± 1.13 | 30.45 | <0.01 |
| Day 4 | 22.93 ± 1.67 | 13.54 ± 1.01 | 26.35 | <0.01 |
| Day 5 | 14.20 ± 1.58 | 8.27 ± 0.28 | 20.75 | <0.01 |
| Day 6 | 3.39 ± 0.38 | 3.84 ± 0.06 | 6.40 | <0.01 |
| Day 7 | 2.51 ± 0.30 | 3.26 ± 0.27 | 10.42 | <0.01 |
| Day 8 | 2.50 ± 0.13 | 3.84 ± 0.07 | 49.07 | <0.01 |
| Day 9 | 2.45 ± 0.15 | 3.82 ± 0.18 | 32.45 | <0.01 |
| Day 10 | 2.20 ± 0.38 | 4.80 ± 0.11 | 35.99 | <0.01 |

2.2. Comparison of the adverse reactions: in the withdrawal symptoms. The difference between the two groups in the adverse reactions, including the abdominal pain, diarrhea, runny nose and tears, was not statistically significant (χ²=0.97, p>0.05), as shown in the Table 3.

TABLE 3
Comparison of the adverse reactions (x ± s)
Adverse Reactions

| | | Abdominal | | Runny | |
|---|---|---|---|---|---|
| | Diarrhea | pain | Chills | nose | Tears |
| The test group | 4 | 7 | 8 | 8 | 10 |
| The Methadone | 3 | 6 | 7 | 8 | 8 |
| control group | | | | | |

2.3 Emotional changes: measured by HAMA, the anxiety symptoms between the test group and the methadone control group on day 1 had no significant statistical difference (p>0.05). On day 5 and day 10, the differences between the two groups were both statistically significant. The anxiety symptom of the test group was significantly lower than that of the methadone control group, as shown in Table 4.

TABLE 4
The emotional changes between the two groups during the
treatment measured by HAMA (x ± s)

| | Clonidine | | | |
|---|---|---|---|---|
| | information | | | |
| | intervention | Methadone | | |
| | agent test | control | | |
| Treatment time | group | group | t | P |
| Day 1 | 18.48 ± 0.86 | 19.24 ± 0.56 | 3.13 | >0.05 |
| Day 5 | 5.03 ± 0.66 | 8.83 ± 0.45 | 24.51 | <0.01 |
| Day 10 | 1.13 ± 0.39 | 3.22 ± 0.36 | 20.32 | <0.01 |

2.4 Side effects: the methadone control group had side effects of dizziness, malaise, nausea, sweating and heart palpitations. The test group had none.

According to an embodiment, the clinical use of the clonidine information intervention agent 9 for smoking cessation is described below.

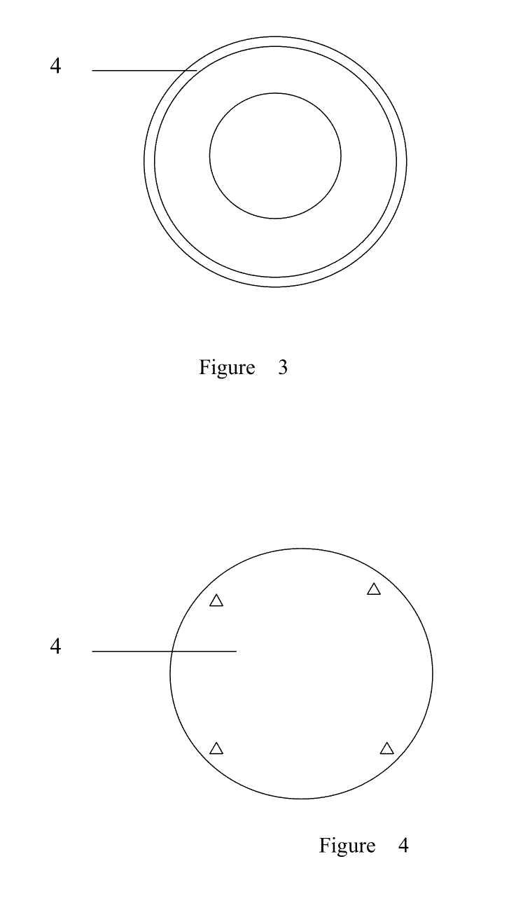

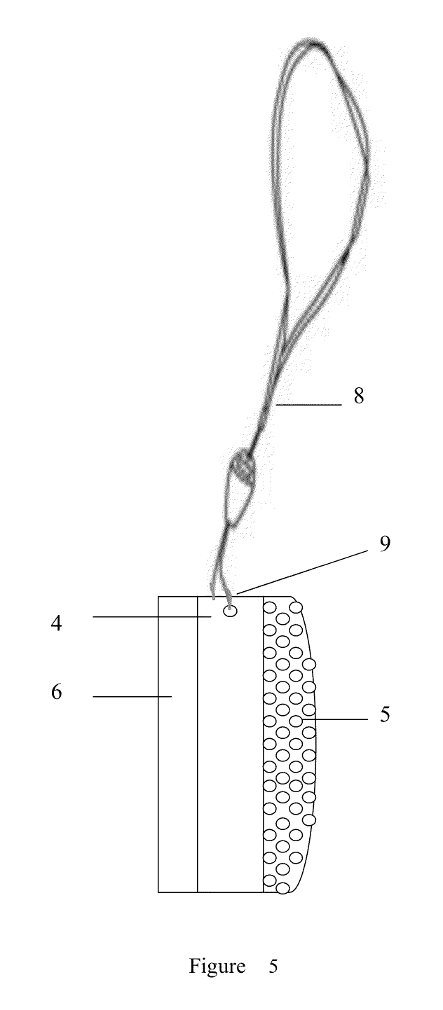

Referring to FIGS. 3-5, the clonidine information intervention agent 9 was prepared according to the following recipe. Putting 60 grams of clonidine hydrochloride raw materials (content 99%), paraffin and 30 grams of wax (particle) into a dispensing machine; packing the mixture of clonidine hydrochloride raw materials and paraffin in 30 air-permeable non-woven fabric bags at a net weight of 3 grams of mixture per bag; sealing the bags by heating to form clonidine information package filler 3. Preparing 30 magnetic suspension containers 4. Magnetic suspension containers are prepared by a method comprising: making a flat circular container 4 box with a diameter of 4.5 cm and a height of 1 cm by plastic injection molding, a lower wall of the container 4 having pores 5 with diameters of 0.1 cm, the flat circular container 4 having an interior void space; inserting a disk-shaped magnet with a magnetic field of 500-1500 Gauss into the void space; covering the void space with a plastic cover 6; the plastic cover having a space to fit another disk-shaped magnet having a magnetic field of 500-1500 Gauss therein; wherein the magnet in the void space and the magnet in the plastic cover 6 are configured to secure the plastic cover 6 on the container 4 by magnetic attraction force. A set of teeth may be arranged to secure the plastic cover 6 to the container 4. The clonidine information package filler 3 may be packed into the magnetic suspension container by opening the plastic cover 6; placing a clonidine information package filler 3 into the void space; closing the plastic cover 6; then sealing the plastic cover 6 with an adhesive to form the clonidine information intervention agent 9.

Clinical use of the clonidine information intervention agent 9 for smoking cessation is compared with the nicotine replacement method.

1. Materials and Methods.

1.1. Clinical Data.

Participants Inclusion Criteria:

A history of smoking of more than 1 year; smoking 15 or more cigarettes per day; age between 18-65 years; no serious liver or kidney dysfunction; and Fagstram score greater than 5 points.

Participants exclusion criteria: a smoking history of less than 1 year; smoke less than 15 cigarettes per day; older than 65 years of age; having serious liver and kidney dysfunction, Fagstram score less than 5 points, having serious skin allergies, alcohol dependence, not being able to accommodate follow-up visits. Further exclusion criteria: first week of treatment adherence is less than 70% (the amount was calculated using the NRT patch), refusing to continue in smoking cessation.

1.2 Research Methods

60 participants who met the participants inclusion criteria were randomly selected. Routine physical examinations were performed before the treatment to record the blood pressure, heart rate, weight, etc. A Fagstram score was given to each participant and confidence in smoking cessation was assessed. The 60 participants were divided into Group A and Group B, 30 in each. Group A was treated with NRT (Nicotine Replacement Therapy) alone. Group B was treated with the clonidine information intervention agent 9, once daily for 6-8 hours each time during the day, by hanging the container 4 on underwear at the umbilicus with the pore 5 side facing the skin. The NRT treatment used nicotine patches (that meet standards for drugs). The initial dose was estimated based on past amount of daily cigarette use, starting with one nicotine patch for each one pack of cigarette used per day. The dose was increased until the participant ceased to have desire for cigarettes. The dose was then maintained for the next 10-14 days. For each next 10-14 days, the dose was reduced by half from the dose for the previous 10-14 days. The treatment stopped when the dose reaches ¼ or ⅛ of the initial dose. Both groups were surveyed in the first, second, fourth, sixth, eighth and twelfth weeks by telephone and in-person visit. The abstinence rate, amount of cigarette use, and body weight were recorded for the first, second, third, fourth, fifth, sixth, seventh, eighth weeks. The abstinence rate and relapse rate of the seventh and twelfth weeks were used in the analysis below. Abstinence rate is calculated based on the standard that no cigarette was smoked in absence of both the clonidine information intervention agent 9 and the NRT treatment. Relapse rate was calculated as restarting smoking after achieving smoking cessation.

1.3. Statistical Analysis

The number of smoking cessation and the abstinence rate of the seventh and twelfth weeks in each group were analyzed statistically. The statistical analysis can be done with using any suitable software such as SPSS. T test was used for continuous data; Chi-square test was used for discrete data. The statistical significance of the difference between the two groups was determined at α=0.05. Non-parametric method-Wilcoxon rank sum test was used for ranking data.

2. Result.

2.1. General characteristics of the participants: 2 females and 58 males. A total of 7 participants in Group A and 5 in Group B dropped out of the study for various reasons. 23 participants in Group A and 25 in Group B finished the smoking cessation study. There were no significant differences between the two groups in terms of age, length of smoking history, number of cigarettes smoked per day, Fagstram score, weight and heart rate before smoking cessation, as shown in Table 5.

2.2 Comparison of the abstinence rate and relapse rate between the two groups: the abstinence rates of Group A participants at week 7 and week 12 were 52.17% ( 12/23) and 34.78% ( 8/23), respectively; the abstinence rates of group B participants at week 7 and week 12 were 64.00% ( 16/25) and 52.00% ( 13/25), respectively. The abstinence rates of Group B is significantly higher than that of Group A, with the statistically significant differences (p<0.05). The relapse rates at week 12 were 33.33% ( 4/12) in Group A, and 25.00% ( 3/16) in Group B, respectively. The relapse rate of Group A was significantly higher than that of Group B (p<0.05), as shown in Table 6.

2.3. Comparison of abstinence symptoms and body weight during the abstinence process: major abstinence symptoms of the two groups include: irritability (35.4%), discomfort with placement of hands and feet (20.83%), muscle or joint pain (18.75%), full body discomfort (18.7%), and gum discomfort (80%). Most of these symptoms occurred during the first three days of the abstinence process. About 80% of the participants experienced weight gain, with an increase in the range of 0.5-3 kg.

TABLE 5
General cessation of smoking cessation participants

| | | | | Number of | | |
|---|---|---|---|---|---|---|
| | Number of | | | cigarettes | | |
| Description | participants | Age | Fagstram | per day | Body weight | Heart rate |
| Group A | 23 | 43.56 ± 13.45 | 7.00 ± 1.90 | 23.03 ± 9.42 | 62.34 ± 16.21 | 75.46 ± 6.23 |
| Group B | 25 | 43.23 ± 13.21 | 7.10 ± 1.81 | 22.46 ± 9.10 | 63.00 ± 15.94 | 75.63 ± 6.53 |
| P | | >0.05 | >0.05 | >0.05 | >0.05 | >0.05 |

TABLE 6
Comparison of the abstinence rate and relapse rate of
the participants

| | Abstinence | Abstinence | Relapse |
|---|---|---|---|
| | rate at week 7 | rate at week 12 | rate at week 12 |
| | (%) | (%) | (%) |
| Group A | 52.17 (12/23) | 34.78 (8/23) | 33.33 (4/12) |
| Group B | 64.00 (16/25) | 52.00 (13/25) | 25.00 (3/16) |
| P | <0.05 | <0.05 | <0.05 |

TABLE 7
Abstinence effects during the treatment and comparison of body weights

| | | | Discomfort | | | | | |
|---|---|---|---|---|---|---|---|---|
| | | | with placement | | | | | |
| | | Full body | of hands and | Muscle or joint | | | | |
| | Palpitation | discomfort | feet | pain | Light sleep | Early wakeup | Irritation | Weight gain |
| Group A | (3) | 1.30 ± 0.76 | 1.24 ± 0.65(6) | 4 | 0 | 0 | 1.02 ± 0.75(8) | 1.77 ± 1.37(20) |
| Group B | (4) | 1.26 ± 0.63(4) | 1.34 ± 0.55(4) | 5 | 0 | 0 | 1.01 ± 0.72(9) | 1.65 ± 1.34(18) |
| | 14.6% | 18.7% | 20.83 | 18.75% | 0 | 0 | 35.4% | 80% |
| P | | | >0.05 | | | | >0.05 | >0.05 |

We claim:

1. An application of compound clonidine hydrochloride, characterized by a use in preparation of an information intervention agent, wherein the information intervention agent is a non-chemical treatment drug; said drug does not contact human body directly during treatment; said drug does not enter human body by absorption; and said drug regulates functions of human body by information of Clonidine Hydrochloride in a physical intervention method in order to achieve treatment effects.

2. An application of claim 1, wherein the information intervention agent is prepared by: mixing clonidine hydrochloride raw material powder with paraffin and supplement carrier materials; packing an amount of the mixture into a bag made of a porous carrier material, the amount predetermined according to needs of the treatment; sealing the bag by heating to make a clonidine hydrochloride information package filler; packing the clonidine hydrochloride information package filler into a container having pores; placing the container near human skin, with the pores facing the human skin; wherein the treatment is achieved through information of clonidine hydrochloride.

3. An application of claim 1, further characterized by a use of the information intervention agent in a secondary application treatment as a chemical treatment drug, or as a synergist agent.

4. A method of preparation of compound clonidine hydrochloride into an information intervention agent, comprising: weighing clonidine hydrochloride raw material, paraffin, and supplement carrier materials at 1-90 weight %, 1-90 weight % and 1-30 weight %, respectively; mixing the clonidine hydrochloride raw material, paraffin and supplement carrier materials; dispensing the mixed clonidine hydrochloride raw material, paraffin and supplement carrier materials into a porous carrier material bag; sealing the bag by heating, to form a clonidine information package filler; packaging the clonidine information package filler into a container having pores; attaching hanger hardware to the container; wherein said supplement carrier materials comprise tourmaline particles.

5. A method of preparing compound clonidine hydrochloride into an information intervention agent, according to claim 4, further comprising: mixing clonidine raw material and supplement carrier materials into molten paraffin to form a mixture; heating the mixture; wrapping the mixture with a porous carrier material; compressing the wrapped mixture in a mold press; cooling the mixture; packaging the mixture into a container having pores.

6. A clonidine hydrochloride information intervention agent, comprising a clonidine information package filler, a container having pores; wherein the clonidine information package filler comprises porous carrier material.

7. A clonidine hydrochloride information intervention agent according to claim 6, wherein the porous carrier material is selected from the group consisting of porous nonwoven fabric, porous fabric, porous paper and porous plastic.

8. A clonidine hydrochloride information intervention agent according to claim 6, further comprising hanger hardware mounted on the container.

9. A clonidine hydrochloride information intervention agent according to claim 6, wherein the container is selected from the group consisting of a box and a bag.
